(19) United States
(12) Patent Application Publication
(10) Pub. No.: US 2022/0408874 A1
(43) Pub. Date: Dec. 29, 2022

(54) WEARABLE PROTECTIVE LAYER TO PRESERVE INSOLE

(71) Applicant: Wearable Shoe Tree, LLC, Frisco, TX (US)

(72) Inventor: Paul SIRAGUSA, Frisco, TX (US)

(21) Appl. No.: 17/358,278

(22) Filed: Jun. 25, 2021

(51) Int. Cl. A43B 7/00; A43B 17/10; A61L 2/232

(52) U.S. Cl. CPC A43B 7/00; A43B 17/10; A61L 2/232

(57) ABSTRACT

A system and method and apparatus comprising an anti-microbial, self-adhesive film, are provided which prevents dirt, sweat, fungus, and bacteria, from absorbing into the insole and the sides and bottoms of the insole. The antimicrobial, self-adhesive film of the invention is provided in a size suitable for application to an insole so as to prevent dirt, sweat, bacteria, foot wort fungus etc. from absorbing into an insole and sides, to and bottom areas of said insole.

### CROSS REFERENCE TO RELATED APPLICATIONS

This application claims the benefit of U.S. Provisional Application Nos. 63/043,855, filed Jun. 25, 2020 and U.S. Provisional Application 63/043,855, filed Jun. 25, 2020, the content of the entirety of which is explicitly incorporated herein by reference and relied upon to define features for which protection may be sought hereby as it is believed that the entirety thereof contributes to solving the technical problem underlying the invention, some features that may be mentioned hereunder being of particular importance.

### IDENTIFICATION OF PARTIES CONCERNED

The Applicant of the present intellectual property matter is Wearable Shoe Trees, LLC of Texas, United States. The inventor of the invention described in this patent documentation is Paul Siragusa of Texas, United States. At the time of filing, John B. Moetteli and the firm Da Vinci Partners LLC of Switzerland represent the Applicant.

### COPYRIGHT & LEGAL NOTICE

A portion of the disclosure of this patent document contains material which is subject to copyright protection. The Applicant has no objection to the facsimile reproduction by anyone of the patent document or the patent disclosure as it appears in the Patent and Trademark Office patent file or records, but otherwise reserves all copyright rights whatsoever. Further, no references to third party patents or articles made herein is to be construed as an admission that the present invention is not entitled to antedate such material by virtue of prior invention.

### BACKGROUND OF THE INVENTION

This invention solves at least the following two problems: (1) Most athletic sneakers have very rare and exclusive “insoles” that only come in the foot ware when you buy the sneakers. Once the insoles are dirty, and stinky they cannot be replaced with the same insole because Shoe/Sneaker brands/manufacturer typically do not sell the insoles separately. Having used, dirty insoles deplete the value of the sneaker and for sneaker traders and resellers this is a significant problem. This invention preserves the original insole that comes in the sneakers and protects it from dirt, bacteria, sweat, odors, viruses etc . . . ; and (2) A Logo on the insole that identifies the brand or style gets worn off after so many wearings. This also depletes the value of the foot-ware and ruins the insole or at least limit this unwanted absorption.

### SUMMARY OF THE INVENTION

A system and method and apparatus comprising an anti-microbial, self-adhesive film, are provided which prevents dirt, sweat, fungus, and bacteria from absorbing into the insole and the sides and bottoms of the insole. The antimicrobial, self-adhesive film of the invention is provided in a size suitable for application to an insole so as to prevent dirt, sweat, bacteria, foot wort fungus etc. from absorbing into an insole, through its sides, top and bottom surfaces.

### BRIEF DESCRIPTION OF THE DRAWINGS

The attached drawings represent, by way of example, different embodiments of the subject of the invention.

FIG. 1 is a schematic view of an exemplary an exclusive “NIKE” sneaker with an insole that cannot be replaced with a new insole.

FIGS. 2A and 2B are schematic views comparing one shoe which benefits from the device of the invention and one that doesn't.

FIG. 3 is a partial, cross-sectional view of the film of the invention.

FIG. 4 is a top view of an insole where the upper portion thereof had no protective film/layer and the lower portion did have protective film/layer of the invention, this view showing the protective film or layer after removal.

FIG. 5 shows the result of an experiment in which is clear that the portion without the protective film, above, is soiled as compared with the portion protected with protective film.

FIGS. 6A and 6B are schematic top views of the invention showing one of many possible variants including application tabs.

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended to distinguish between similar elements and not necessarily for describing a sequential or chronological order. Moreover, relative terms like ‘front’, ‘back’, ‘top’ and ‘bottom’, and the like in the Description and/or in the claims are not necessarily used for describing exclusive relative position. Those skilled in the art will therefore understand that such terms may be interchangeable with other terms, and that the embodiments described herein are capable of operating in other orientations than those explicitly illustrated or otherwise described.

### DETAILED DESCRIPTION OF THE PREFERRED EMBODIMENT

The following description is not intended to limit the scope of the invention in any way as it is exemplary in nature, serving to describe the best mode of the invention known to the inventors as of the filing date hereof. Consequently, changes may be made in the arrangement and/or function of any of the elements described in the exemplary embodiments disclosed herein without departing from the spirit and scope of the invention.

Most shoes come with insoles and most insoles are made from a soft absorbent material. Especially rare and expensive footwear like “NIKE”™, “YEEZY”™, etc. come with their own branded insoles that are not replaceable unless you buy another pair of the same sneakers. Some of these sneakers can cost up to $10,000 USD, so they can be very expensive. When the insole absorbs sweat, bacteria, dirt and other elements, it causes your shoes to hold germs, look used and dirty and worst of all, stink.

The original equipment manufacturer (OEM) insoles that come in all “NIKE”™, “JORDAN”™'s and “YEEZYS”™ exclusive footwear for example, are not produced for separate sale. Consequently, you can't replace the insoles once they are dirty and looking worn out and old. This devalues the sneaker or footwear.

The invention offers the ability to protect the insole so as to:

1. preserve a logo on the insole from rubbing off from wearing your shoes;

2. keep your insoles looking new and germ free; and

3. preserve them while being worn, all while keeping them on an essentially germ-free anti-microbial path thus maintaining healthy shoes (and therefore also feet) for months and years to come.

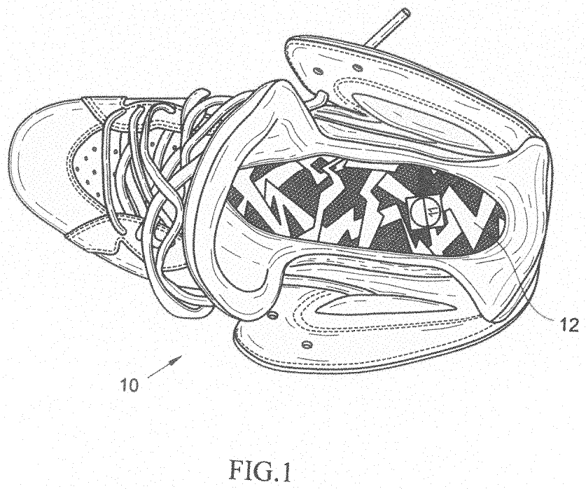

Referring now to FIG. 1, an exemplary embodiment of an exclusive “NIKE”™ sneaker 10 with an insole 12 that cannot be re purchased is shown. It's a must to keep the insoles clean and looking new because, practically speaking, one cannot replace the insoles. The various insoles 12 that come in footwear essentially all include important identifying logos 14, typically on the heel area or in their center area. These logos 14 help identify the brand and often are critical in determining whether the footwear is authentic. If these logos can be maintained as good as new, this solves a huge problem and helps preserve the irreplaceable and valuable insole.

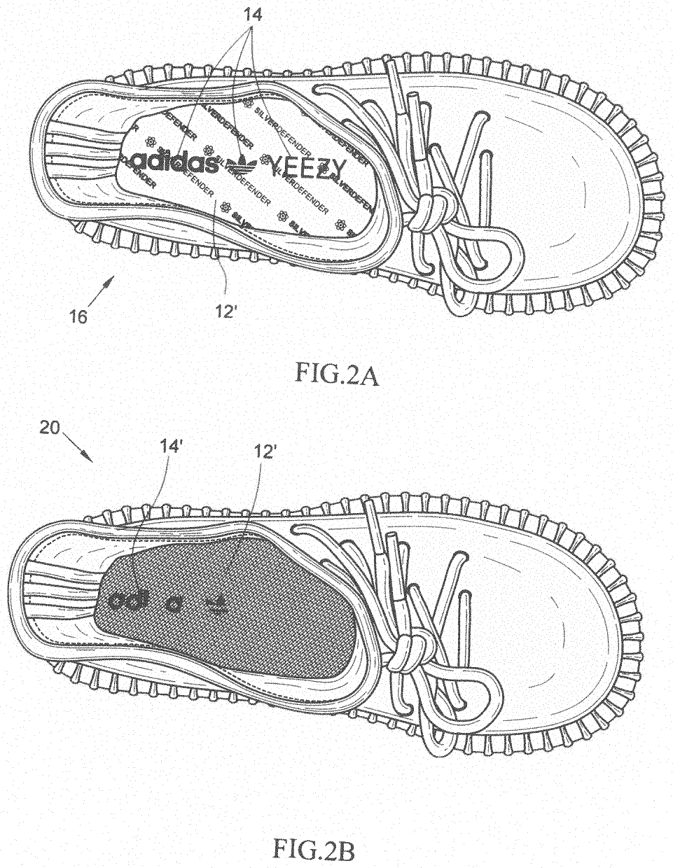

Referring now to FIGS. 2A and 2B, show a comparison of one shoe 16 with the film 18 of the invention (FIG. 2A) and one shoe 20 without (FIG. 2B). These insoles are both white and have the same exact black Logo 14 on them. It is apparent from the figure that the logo on the bottom sneaker 20 has been rubbed off and it is very dirty. In contrast, the top sneaker 16 still has the brand name 14 and is being preserved and protected. Such logos and brand names 14 identified on these insoles 12′ are very important, but unfortunately start to wear off after just several wears and eventually completely fade away, if not protected by the present invention.

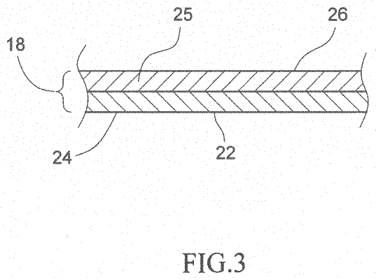

Referring now to FIG. 3, the film 18 of the invention is made up of a plastic layer 25 to which an adhesive backing layer 24 is applied, optionally with special treatment of the plastic layer 25 to promote adhesion.

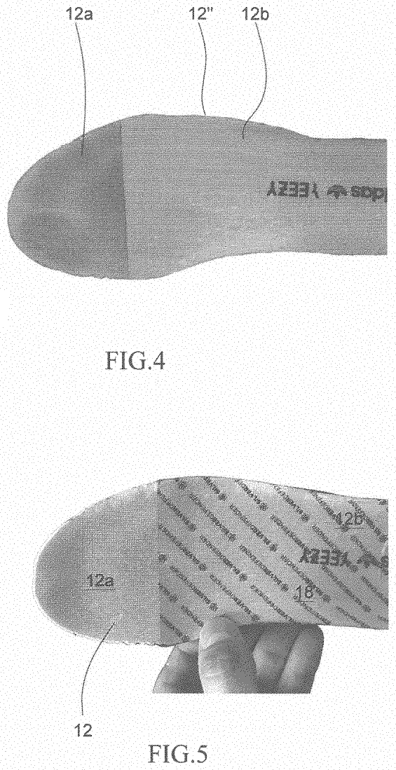

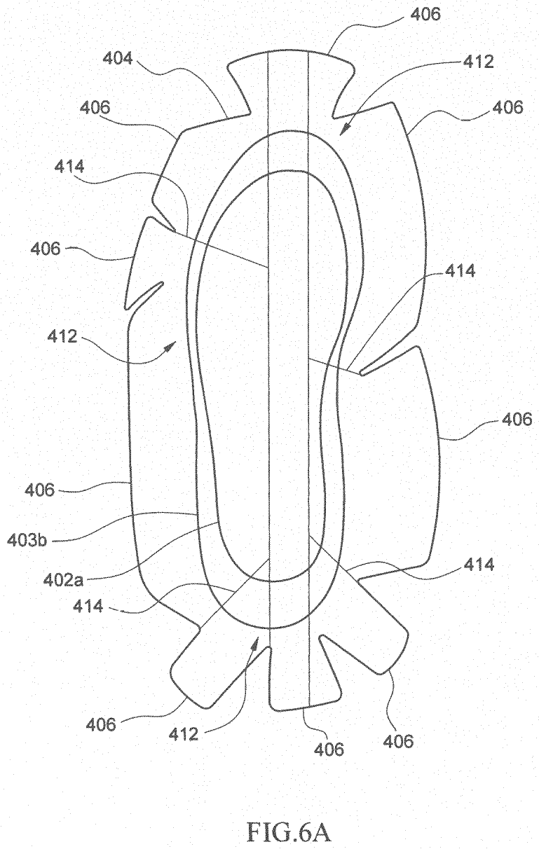

Referring now to FIG. 4, an insole 12″ is shown where the upper portion 12a did not benefit from the protective film 18 (shown in FIG. 5) of the invention and the lower portion 12b did. In this view, the protective film 18 is removed to show the contrast. Clearly, the upper portion 12a of the insole is soiled, and the lower portion 12b is clean, as new.

Referring now to FIG. 5, the result of an experiment is illustrated. In an experiment, the thin anti-microbial adhesive film combination 18 was wrapped around only to a part of the insole 12. Only after one week of wearing the shoes, one can already see how much dirt and germs (see area 12b) it's protecting the insole 12 from absorbing. The thin, anti-microbial adhesive film 18 wraps insoles 12 to preserve them and protect them from the elements listed above and provide a safer, healthier environment for both the wearer's foot and the footwear. They are user friendly and easy to install. These will especially be appreciated by consumers who share, sell, trade sneakers/shoes.

The device 18 according to the invention is made from a special plastic film that is extremely strong, stretchy, durable and made with an anti-microbial material.

In one embodiment, one side 22 (the interior side 22 directed towards the insole 12 when installed on the insole) incorporates an adhesive to enable the film to stick to and therefore cover and protect insoles. The adhesive 24 is a fixation means that provides as the effect that the plastic layer 25 does not dislodge or move out of place while the sneaker/shoe is being used. The other side 26 (the side directed towards the wearer's foot when worn) of the device 18 may be smooth, textured (for “non slip”/stop from sliding in the shoe), or slotted or perforated (to permit breathing/air flow).

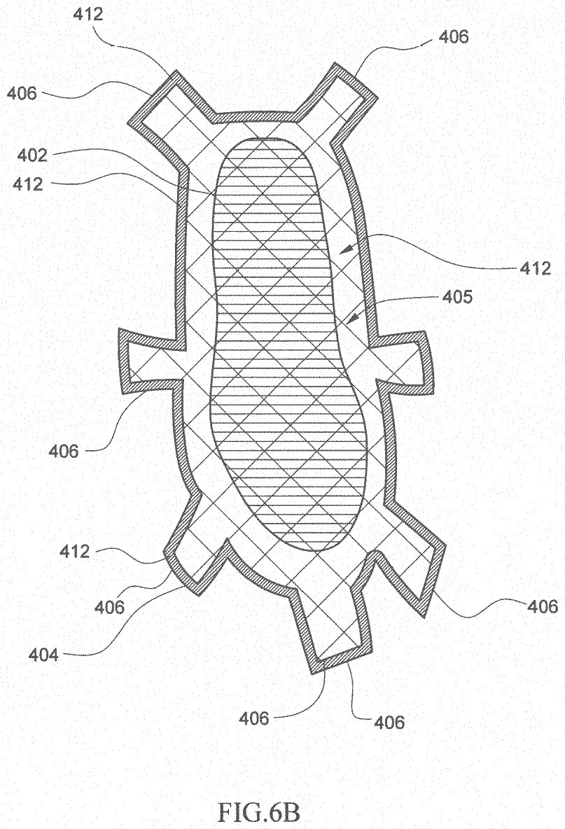

Referring now to FIGS. 6A and 6B, schematic top views of the invention are shown. For example, an insole 402 is shown. In FIG. 6A, for example, two different sizes of insoles 402a, 402b are shown in order to visualize the applicability of the invention of a certain size to different sizes of insoles. As described above, the device according to the invention is made from a special plastic film that is extremely strong, stretchy, durable and made with an anti-microbial material. In a preferred embodiment, the outer contour of the film 404 overlaps the outer contour of the insole when placed on the insole. In another preferred embodiment, the outer contour of the films comprises flaps 406 that facilitate installation. The border area 410 along the outer contour of the film 404 can be sticky for easy application but not too sticky so as to facilitate easy removal. The sticky border area is configured to seal to the bottom of the insole when installed in order to prevent odors.

In one embodiment, the side of the film 404 directed towards the insole when installed on the insole is free of adhesive in the area where it gets in contact with the upper part of the insole, so it does not leave any sticky residue in the insole when it is removed and any logo/pattern/etc. present on the upper part of the insole will not be pulled off when the film is removed from that insole. However, in another embodiment, said area may be covered, either partially or fully, with adhesive. Exemplary, said area may be as large as the crossed area 405 in FIG. 6B. In one preferred embodiment, the film comprises extra margins 412 overlapping the outer contour of the insole, such to be securely foldable over the bottom of the insole. This embodiment further provides, optionally, cuttings 414, allowing portions of the film to overlap or to strut apart from each other when folded over the bottom of the insole. The cuttings shown in FIG. 4A are shown at preferred positions, however, they may be as well on other positions. Co-branding on the film is possible such that on one hand the logo/pattern/etc. of the insole is visible to an observer, and on the other hand a logo/pattern/etc. may be present on the film.

Pine tree rosin mixed with a polymer can be used as a natural shield against harmful microbes. See products offered by PREXELLENT (see prexellent.com). In addition, 3M™ Durable Protective Film 7750AM using a semi-permanent adhesive (Part Number 7750AM, 3M Product Number 7750AM, 3M ID 70000244056) may also be used. The protective film is typically 2 mil thick with an adhesive for semi-permanent applications includes antimicrobial additive. The film is optionally transparent with a hard-coat layer that resists scratches, abrasion and chemicals. It is also resistant to various common cleaning products such as bleach, isopropanol and hydrogen peroxide. The PET film provides robust protection with high clarity. It uses 3M™ Adhesive 400: good wet-out and clarity, non-yellowing when exposed to UV light. It includes an EPA-registered antimicrobial agent*. The EPA-registered silver ion antimicrobial additive* helps the hard-coat resist the growth of microbes, odour-causing bacteria, mould and mildew between cleanings. The silver ion antimicrobial agent that protects the film is integrated into the structure of the hard-coating and will reasonably last the lifetime of the coating. This product uses 3M™ Adhesive 400, which offers good wet-out and clarity and is non-yellowing when exposed to UV light; it is intended for permanent to semi-permanent applications. The hard-coat provides protection against scratches, abrasion and cleaning chemicals for surfaces in high-touch environments and, in addition, inhibits most bacteria, mould and mildew on the hard-coat between cleanings. Note that full protection against microbes and pathogens is impossible. Therefore, the user must take additional measures to better ensure their safety against contagious viruses such as COVID-19. Note that 3M™ Durable Protective Film 7760AM is specifically made for removable applications such as required by the invention.

A suitable anti-microbial film is preferably treated in areas where an adhesive film is to be provided via an abrasion treatment, a corona light treatment, or other treatment suitable for increasing the adherence of the adhesive to the film, in order to avoid that it comes off on the insole, separating from the plastic anti-microbial film due to the abrasion caused by foot movement in the shoe. Separation causes the film to fail to protect the insole. After the adhesive film is provided, a peel-off protective tab is applied to the adhesive (such as exists with band-aids) to allow exposure of the adhesive at the time chosen by the user, just prior to applying the adhesive film to the foot, the insole, or the shoe.

Another suitable anti-microbial film can be found on: silverdefender.com. Installation is Simple. First, make sure the surface to be protected is clean and dry. Second, peel and stick. Optionally, application instructions are printed on the protective film covering the adhesive. These instructions therefore are removed from the adhesive backed sheet and so available for reference when installing the film. Optionally, the adhesive on the film is pressure sensitive. Make sure to press down thoroughly, including all the edges for optional adhesion.

The present invention is suitable for various types of footwear. From sneakers or shoes with insoles to sandals or flip flops. As is clear, one can also apply the invention to the tops of flip flops or sandals to protect and help keep the top area clean, new and preserved.

A detailed description on how the invention works is now provided. The invention is typically die-cut and may be substantially of a rectangular shape or cut to an insole shape. The film should be wider and longer than the actual “insole” so that the excess portion can be wrapped around the insole from the top of the insole then wrapping around to the bottom of the insole to optionally create an air tight fit. A user simply peels off the backing protecting the adhesive and carefully applies the adhesive side to the insole. The firm can be flattened out with the user's fingers so there are no air bubbles in it. Then wrap the excess film around the insole. For sandals or flip flops, there is optionally provided a dotted perforated line in between the big toe and pointer toe to allow for the piece of the sandal that is often disposed in between those specific toes to pass through. This feature may be pre made at the factory or the consumer may simply cut the film of the invention at the appropriate location to accommodate that piece of the sandal.

Note that it is recommended, for example, to replace the film of the invention every 90 days or after 90 wearings. However, more frequent replacement results in better protection.

The invention can be summarized as including the following features sets:
1. An antimicrobial, self-adhesive film provided in a size suitable for application to an insole so as to prevent dirt, sweat, bacteria, foot wort fungus etc. from absorbing into sides, top and at least a portion of the bottom areas of said insole.
2. The film of feature set 1, wherein the dirt comprises black spots and wherein such black spots are in particular prevented on the top surface of the insole, in particular in the heel portion.
3. The film of feature set 1 or 2, wherein the film is adapted to prevent any logos/markings or the like present on an insole from rubbing off while the insole is installed in a shoe such as a sneaker and the shoe is worn.
4. The film of any of above feature sets, wherein the film is adapted to block the reading of germs.
5. The film of any of above feature sets, wherein the film is slotted and/or perforated to allow air flow through the device.
6. A film of any of the above feature sets, wherein the film comprises various textures, from a smooth surface to a more textured “non slip” surface for people who may be wearing their shoes without socks, the textured surface would offer a non-slip texture finish to the surface of the device and give the wearer a more secure non slip feeling.
7. The film of feature set 1 adapted to be easily be wiped down and cleaned in between wearings with a damp cloth to maintain a safe environment for the next person who may wear that shoe, such as when applied to insoles of bowling shoes, rental skies, ice skates or roller skates, for example, thereby helping to stop the spread of foot fungus like foot warts and Athlete's foot.
8. The film of feature set 1, adapted to be easily replaceable with a new one by simply taking off the old one and applying the new one, by just peeling the backing of the anti-microbial sticker paper off and applying to the insole, wrapping over laying extra portion around the perimeter underneath the insole and sticking it to the bottom of the insole, to ensure no bacteria penetrates the insole from the top, sides or bottom of the insole.
9. The film of feature set 1, adapted for people who suffer from stinky feet, wherein the antimicrobial film is scented to help prevent odours from forming inside footwear.
10. The film of feature set 1, wherein the film is larger than the insole so that it may be wrapped around all the way under the insole to not only stay in place by virtue of the self-adhesive, but also to keep moisture out of the sides and bottoms of the insole, thus keeping shoes essentially germ free.
11. The film of feature set 10, wherein the insole is combined with a plastic bag or pouch with sealing means wherein, the insole is adapted to be slipped into the plastic bag or pouch and sealed therein.
12. The film of feature set 11, wherein the sealing means is an adhesive edge which can be sealed against an adjacent edge.
13. The film of feature set 11, wherein the sealing means is a zip-lock type interlocking sealing edge pair.

In an advantage, the film of the invention helps prevents dirt, sweat, fungus, and bacteria from absorbing into the insole and the sides and bottoms of the insole.

In another advantage, the insoles are protected from becoming soiled and therefore maintain a fresh, new look longer.

In another advantage, sandals protected with the film of the invention can be worn with or without socks.

It should be appreciated that the particular implementations shown and herein described are representative of the invention and its best mode and are not intended to limit the scope of the present invention in any way.

As will be appreciated by skilled artisans, the present invention may be embodied as a system, a device, or a method.

Moreover, the system contemplates the use, sale and/or distribution of any goods, services or information having similar functionality described herein.

The specification and figures should be considered in an illustrative manner, rather than a restrictive one and all modifications described herein are intended to be included within the scope of the invention claimed. Accordingly, the scope of the invention should be determined by the appended claims (as they currently exist or as later amended or added, and their legal equivalents) rather than by merely the examples described above. Steps recited in any method or process claims, unless otherwise expressly stated, may be executed in any order and are not limited to the specific order presented in any claim. Further, the elements and/or components recited in apparatus claims may be assembled or otherwise functionally configured in a variety of permutations to produce substantially the same result as the present invention. Consequently, the invention should not be interpreted as being limited to the specific configuration recited in the claims.

Benefits, other advantages and solutions mentioned herein are not to be construed as critical, required or essential features or components of any or all the claims, herein incorporated by reference.

As used herein, the terms “comprises”, “comprising”, or variations thereof, are intended to refer to a non-exclusive listing of elements, such that any apparatus, process, method, article, or composition of the invention that comprises a list of elements, that does not include only those elements recited. Unless otherwise explicitly stated, the use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or adapted by the skilled artisan to other designs without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

Additional features and functionality of the invention are described in the claims appended hereto and/or in the abstract. Such claims and/or abstract are hereby incorporated in their entirety by reference thereto in this specification and should be considered as part of the application as filed.

Multiple variations and modifications are possible in the embodiments of the invention described here. Although certain illustrative embodiments of the invention have been shown and described here, a wide range of changes, modifications, and substitutions is contemplated in the foregoing disclosure. While the above description contains many specific details, these should not be construed as limitations on the scope of the invention, but rather exemplify one or another preferred embodiment thereof. In some instances, some features of the present invention may be employed without a corresponding use of the other features. Accordingly, it is appropriate that the foregoing description be construed broadly and understood as being illustrative only, the spirit and scope of the invention being limited only by the claims which ultimately issue in this application.

What is claimed is:

1. An antimicrobial, self-adhesive film provided in a size suitable for application to an insole so as to prevent dirt, sweat, bacteria, foot wort fungus etc. from absorbing into the sides, top and at least a portion of the bottom areas of said insole.

2. The film of claim 1, wherein the dirt comprises black spots and wherein such black spots are in particular prevented on the top surface of the insole, in particular in the heel portion.

3. The film of claim 1, wherein the film is adapted to prevent any logos/markings or the like present on an insole from rubbing off while the insole is installed in a shoe such as a sneaker and the shoe is worn.

4. The film of claim 1, wherein the film is adapted to block the reading of germs.

5. The film of claim 1, wherein the film is slotted and/or perforated to allow air flow through the device.

6. A film of claim 1, wherein the film comprises various textures, from a smooth surface to a more textured “non slip” surface for people who may be wearing their shoes without socks, the textured surface would offer a non-slip texture finish to the surface of the device and give the wearer a more secure non slip feeling.

7. The film of claim 1 adapted to be easily be wiped down and cleaned in between wearings with a damp cloth to maintain a safe environment for the next person who may wear that shoe, such as when applied to insoles of bowling shoes, rental skies, ice skates or roller skates, for example, thereby helping to stop the spread of foot fungus like foot warts and Athlete's foot.

8. The film of claim 1, adapted to be easily replaceable with a new one by simply taking off the old one and applying the new one, by just peeling the backing of the anti-microbial sticker paper off and applying to the insole, wrapping over laying extra portion around the perimeter underneath the insole and sticking it to the bottom of the insole, to ensure no bacteria penetrates the insole from the top, sides or bottom of the insole.

9. The film of claim 1, adapted for people who suffer from stinky feet, wherein the antimicrobial film is scented to help prevent odours from forming inside footwear.

10. The film of claim 1, wherein the film is larger than the insole so that it may be wrapped around all the way under the insole to not only stay in place by virtue of the self-adhesive, but also to keep moisture out of the sides and bottoms of the insole, thus keeping shoes essentially germ free.

11. The film of claim 10, wherein the insole is combined with a plastic bag or pouch with sealing means wherein, the insole is adapted to be slipped into the plastic bag or pouch and sealed therein.

12. The film of claim 11, wherein the sealing means is an adhesive edge which can be sealed against an adjacent edge.

13. The film of claim 11, wherein the sealing means is a zip-lock type interlocking sealing edge pair.
